(19) United States
(12) Patent Application Publication
(10) Pub. No.: US 2005/0136085 A1
(43) Pub. Date: Jun. 23, 2005

(54) Panthenol and natural organic extracts for reducing skin irritation

(76) Inventor: David Bellamy, N. Charleston, SC (US)

Correspondence Address: WELSH & FLAXMAN LLC, 2450 CRYSTAL DRIVE, SUITE 112, ARLINGTON, VA, 22202, US

(21) Appl. No.: 11/014,243

(22) Filed: Dec. 17, 2004

Related U.S. Application Data
(60) Provisional application No. 60/530,655, filed on Dec. 19, 2003.

(51) Int. Cl. A61K 35/78; A61K 7/135; A61K 7/09; A61K 7/13; A61K 31/19

(52) U.S. Cl. 424/401; 424/757; 424/764; 424/769; 8/406; 424/62; 424/760; 424/70.2; 514/557; 514/559

(57) ABSTRACT

Panthenol combined with natural organic extracts are well suited for reducing skin irritation that is normally elicited by exposure to irritant active agents or compounds, particularly found in hair permanent and relaxing compositions, hair colorants and depilatories.

### CROSS REFERENCE TO RELATED APPLICATION

This application is based upon U.S. Provisional Patent Application No. 60/530,655, filed Dec. 19, 2003, entitled “NATURAL ORGANIC EXTRACTS FOR REDUCING SKIN IRRITATION”.

### BACKGROUND OF THE INVENTION

This invention relates to compositions for reducing adverse reactions of the skin to chemicals and compositions that contact the skin, e.g., topical cosmetic and pharmaceutical compositions, and for reducing such adverse reactions caused by certain kinds of physical contact. Exposure of human skin to chemicals contained in topical cosmetic and pharmaceutical compositions can result in adverse reactions to the skin, including irritation response and contact sensitization of the skin. As used herein, the term cosmetic and pharmaceutical compositions are used in the widest sense of the term, and encompass any composition that is applied to the skin for a beneficial effect. As used herein, contact sensitization of the skin refers to adverse systemic immunological reactions of the skin, e.g., itching, burning, swelling, redness or inflammation. Irritation response of the skin involves similar symptoms in which the systemic immune system plays no role.

Certain types of physical contact with human skin can cause irritation response of the skin. For example, the treatment of hair with hair permanent or relaxing solutions or the removal of hair from human skin by waxing methods is known to cause irritation to most, if not all, persons who have permanent, straightening or waxing treatments. Hereinafter, any of these or other types of physical contact which cause irritation responses of human skin are referred to as “contact physical irritants.”

### SUMMARY OF THE INVENTION

An object of this invention is to provide a composition and method for reducing the irritation response and contact sensitization of human skin to chemicals and compositions that contact the skin, especially the scalp, e.g., topical cosmetic and pharmaceutical preparations.

Another object of this invention is to provide a composition and method for reducing the irritation response of human skin to contact physical irritants.

We have discovered that compositions containing Panthenol (D-(+)-2,4-dihydroxy-N-(3-hydroxypropyl)-3,3-dimethylbutyramide) preferably in admixture with one or more extracts selected from the group consisting of: extract of Ruscus Aculeatus, extract of Centella Asiatica, extract of Calendula, extract of Aesculus Hippocastanum and extract of Glycyrrhiza Glabra, in a carrier suitable for use in contact with the skin are useful for reducing adverse reactions of the skin to chemical irritants and to contact physical irritants. Preferably, the composition of the invention comprises at least 2.5% by weight of Panthenol, together with at least 0.1% by weight of the extract of Ruscus Aculeatus, extract of Centella Asiatica, the extract of Calendula, the extract of Aesculus Hippocastanum and the extract of Glycyrrhiza Glabra, individually.

As used herein, the term “carrier” shall include materials that are suitable for use in contact with the skin and which, when combined with the extracts in the composition of the invention, take a form that is suitable for application to the skin, e.g., the form of a liquid, gel, ointment, cream, lotion or the like.

The compositions of the invention may be applied in effective amounts to the skin in any suitable manner at about the time the skin is exposed to a chemical irritant or a contact physical irritant. Preferably, the composition of the invention is applied to the skin in an effective amount simultaneously with (e.g., as part of a composition containing a chemical irritant) the skin being exposed to a chemical or contact physical irritant. It is within the scope of the invention, however, to apply the composition of the invention shortly before (e.g., less than about half an hour before, preferably less than about 15 minutes before) or shortly after (e.g., less than about half an hour after, preferably less than about 15 minutes after) the skin is exposed to a chemical or contact physical irritant.

In use, the composition of the invention prevents the induction of any noticeable adverse systemic immunological reaction of the skin (i.e., it prevents the induction of contact sensitization) in some cases and reduces the extent of contact sensitization in other cases. Similarly, in use the composition of the invention prevents the onset of an irritation response to the skin in some cases and reduces the extent of irritation response to the skin in other cases.

### DETAILED DESCRIPTION OF THE INVENTION

It has been discovered that anti-irritant compositions containing Panthenol in admixture with at least one compound from the group consisting of: the extract of Ruscus Aculeatus, the extract of Centella Asiatica, the extract of Calendula, the extract of Aesculus Hippocastanum and the extract of Glycyrrhiza Glabra and mixtures thereof, in a carrier suitable for use in contact with the skin are useful for reducing adverse reactions of the skin to chemical irritants and to contact physical irritants.

Panthenol is the alcohol form of pantothenic acid, more familiar as Vitamin B5. In a living cell, panthenol is converted to pantothenic acid, which then becomes an important part of the compound “Coenzyme A”, which is important in cellular metabolism. In hair, which contains no living cells, it remains as panthenol. Because it could become a vitamin if it were ever to get to a living cell, it is marketed as a “provitamin”, even though its effects as a vitamin are never used. Because panthenol binds well to hair follicles, and attracts moisture from the air, it is a moisturizing agent used in shampoos and conditioners. Panthenol lubricates the hair without feeling greasy and smoothes roughened hair surfaces, making them shiny and easier to comb.

The anti-irritant composition of the present invention preferably contains at least one additional active ingredient in admixture with the panthenol selected from the group consisting of: extract of Ruscus Aculeatus (Butcher's Broom), extract of Centella Asiatica (Gotu kola), extract of Calendula (Marigold), extract of Aesculus Hippocastanum (Horse Chestnut), and extract of Glycyrrhiza Glabra and mixtures thereof.

In one embodiment of this invention, the anti-irritation composition of the invention contains at least 0.5% to 10% by weight of panthenol in a suitable carrier. Preferably, the composition of the invention contains panthenol in an amount at least 2.5% up to 10% by weight, most preferably 5% by weight of the total anti-irritant composition.

The anti-irritant composition of the invention additionally contains 0% to 20% by weight, preferably at least 0.1% to 20% by weight, most preferably at least 0.1% to 10% by weight, of at least one extract selected from the group of: the extracts of Ruscus Aculeatus, Centella Asiatica, Calendula, Aesculus Hippocastanum, and Glycyrrhiza Glabra, and mixtures thereof. These extracts assist in the reduction of swelling, edema and inflammation caused by irritation producing substances. The composition of the present invention preferably contains at least about 0.1% to about 5% by weight of each of the extracts of Ruscus Aculeatus, Centella Asiatica, Calendula, Aesculus Hippocastanum, and Glycyrrhiza Glabra.

These foregoing amounts of panthenol and extracts and are herein defined as being anti-irritant effective amounts.

A wide variety of materials may be used as carriers or “physiologically acceptable medium” in the formulation of the anti-irritant compositions of the invention, preferably water and/or propylene glycol. By the expression carrier or “physiologically acceptable medium” is intended a medium which is compatible with the skin, mucous membranes (including the inside of the eyelids and the lips), nails and/or keratin fibers (head hair and/or body hairs). In addition, this physiologically acceptable medium contains no reaction by-product or residual product.

Examples of such carriers or medium include hydroalcoholic solutions and water and oil emulsions. In addition, the compositions of the invention may include other active or non-active ingredients for the skin as well as one or more fragrances (usually an irritant), preservatives, surfactants, emulsifiers, and other materials of the type that are conventionally included in cosmetic and pharmaceutical compositions. Among the wide range of active ingredients that may be included in the composition of the invention, but which do not comprise part of the invention, are vitamins other than panthenol, sunscreens, antioxidants, free radical scavengers, lipids, glycolipids, and other materials that have desirable cosmetic or pharmaceutical effects on the skin.

Additional additives to the formulation of the anti-irritant compositions of the invention include Polysorbate 80 (ethoxylated sorbitan monooleate) and polyvinylpyrrolidone in amounts individually preferably ranging from 0.5 to 5% by weight. Polyvinylpyrrolidone assists in desensitizing local areas while Polysorbate 80 is a nonionic surfactant and emulsifier.

The anti-irritant compositions according to the invention are well suited for topical application onto the skin, keratin fibers, nails or mucous membranes; in particular, the subject compositions are cosmetic and/or dermatological compositions.

The amounts of the various constituents of the compositions according to the invention are those conventional in the fields under consideration. The nature of the additives and adjuvants and their amounts should be such that they do not modify the properties of the compounds of extracts according to the invention.

The anti-irritant compositions of the invention can be used as additives for hair care products in the form of solutions, creams, gels, emulsions or mousses, and in particular as hair care compositions such as shampoos, treating lotions, restructuring lotions for the hair including permanents, relaxers and straighteners, lotions or gels for combating hair loss, anti-parasitic shampoos, and the like.

The anti-irritant compositions of the invention can be also used as additives in any composition possessing irritating effects such as, but not limited to: cleansing, protective, treatment or care products and/or as makeup products for facial and/or body skin, mucous membranes and/or keratin fibers. The anti-irritant compositions in particular constitute treatment or care creams for the face, for the hands or for the body (for example day creams, night creams, anti-sun or sunscreen creams), protective or care body milks, lotions (cleansing lotions, anti-sun or sunscreen lotions), skincare mousses or gels, or bath compositions. They can also constitute makeup products, in particular, for the cheeks, the lips, the eyelashes and the eyelids, such as foundations, lipsticks and eyeshadows: to this end, they can contain dyestuffs and in particular chemical dyes or pigments. Many topically applied products have an irritant nature, especially for individuals (users) with easily irritated skin. Thus, these products, as described above, cause stinging, itching or tautness, which results in discomfort to the user, which discourages further use.

The products which can cause an irritation of the skin are typically preservatives, surfactants, fragrances, solvents, propellants and active agents, and mixtures thereof.

Thus, even the products which are considered to be inert in a cosmetic composition can have an irritant nature when they are applied to the skin, the scalp, the nails or the mucous membranes, such as, in particular, preservatives, surfactants, fragrances, solvents or propellants. Products considered as active agents in cosmetic compositions can have an irritant nature when they are applied to the skin, the scalp, the nails or the mucous membranes. This can be referred to as an irritant side effect exhibited by these products, which are, in particular, active agents such as, especially, certain hair relaxing/straightening or permanent-waving active agents (thiols), sunscreens, hydroxy acids, in particular α-hydroxy acids (glycolic, lactic, malic, citric, tartaric, mandelic, etc.) and. β-hydroxy acids, especially salicylic acid and its derivatives, keto acids, in particular in α.- and. β-form, derivatives of hydroxy or keto acids, especially in α.- and β-form, retinoids (retinol and its esters, retinal, retinoic acid and its derivatives, anthralins (dioxyanthranol), anthranoids, peroxides (in particular benzoyl peroxide), minoxidil and its derivatives, lithium salts, anti-proliferating agents such as 5-fluorouracil or methotrexate, certain vitamins such as vitamin D and its derivatives and vitamin B9 and its derivatives, hair tints or dyes (para-phenylenediamine and its derivatives, aminophenols), perfuming alcoholic solutions (fragrances, eaux de toilette, aftershave and deodorants), antiperspirants (some aluminum salts), hair-removing active agents, depigmenting agents (hydroquinone), capsaicin, anti-louse active agents (pyrethrin), ionic and nonionic detergent agents and propigmenting agents (dihydroxyacetone, psoralens and methylangecilins), and mixtures thereof. Preferably, the anti-irritant compositions of the invention may be included in admixture with these compositions containing chemical irritants, thereby permitting the simultaneous application of panthenol and the extracts together with the irritant to the skin. Typically, the panthenol/extract formulation of the present invention in their carriers are mixed in an amount of 1 to 10% by weight, preferably about 5% by weight of the compositions containing chemical irritants. These amounts are herein defined as irritation reduction or prevention effective amounts for reducing or preventing irritation by a cosmetic or pharmaceutical composition containing a chemical irritant. Preferably an irritation reduction or prevention effective amount of the anti-irritant compositions of the present invention is admixed with a cosmetic or pharmaceutical composition containing a chemical irritant, such as a hair relaxer composition or a hair permanent or waving composition, either during the initial formulation of the cosmetic or pharmaceutical composition or as an adjuvant prior to their use.

The anti-irritant compositions of the invention may also be applied to the skin shortly before (about 30 minutes or less, preferably about 15 minutes or less) or shortly after (less than about 30 minutes after, preferably less than about 15 minutes after) a cosmetic or pharmaceutical composition containing a chemical irritant is applied to the skin.

The use of the anti-irritant composition in such a manner reduces the irritation resulting from removing hair by such waxing techniques and depilatories. The composition of the invention may also be applied shortly after (less than about 30 minutes after, preferably less than about 15 minutes after) the skin is waxed for the purpose of removing hair.

The present invention presents the possible advantage of being able to increase the amount of active agents, which are conventionally of irritant nature, in compositions with respect to the amount normally employed, with a view to improved effectiveness of the latter. Thus, the amount of active agent which can be used in the composition advantageously ranges from 5% to 50% of the total weight of the composition. In particular, it is possible to formulate hydroxy acids at up to 50% of the total weight of the composition, salicylic acid or derivatives thereof at up to 10%, or retinoids at up to 5%, without any discomfort for the user.

The following non-limiting Example illustrates a composition of the present invention.

### EXAMPLE 1

A formulation comprising an oil-in-water emulsion was prepared by combining the following ingredients in the sequence and manner described below:

| | Percentage by |
|---|---|
| Ingredient | weight |
| Purified Water | 40 |
| Propylene Glycol | 39.5 |
| Polyvinylpyrrolidone | 5 |
| Polysorbate 80 | 5 |
| Panthenol (D-(+)-2,4-dihydroxy-N-(3- | 5 |
| hydroxypropyl)-3,3-dimethylbutyramide) | |
| Ruscus Aculeatus Extract | 1 |
| Centella Asiatica Extract | 1 |
| Calendula Extract | 1 |
| Glycyrrhiza Glabra | 1 |
| Aesculus Hippocastanum Extract | 1 |
| Methyl Paraben/Propyl Paraben | .5 |

The above ingredients were prepared using the following procedure:
Stir slowly and add ingredients listed below.
Add Methyl Paraben
Add Propyl Paraben
Add Propylene Glycol
Add Panthenol
Add Polysorbate 80
Add Polyvinylpyrrolidone
Add Ruscus Aculeatus Extract
Add Centella Asiatica Extract
Add Calendula Extract
Add Aesculus Hippocastanum Extract
Add Glycyrrhiza Glabra

Extracts may be added as a compound, which is already blended and requires no heat to add to solution.

If all herbs are provided as extracts no heating is required.

Herbs may be a slurry, paste or granules. Extracts do not require heavy filtration or repeated filtration.

Stir slowly for 30 minutes.

Allow finished product to set in ambient temperature for one hour before using.

While the invention has been described in terms of various specific and preferred embodiments, the skilled artisan will appreciate that various modifications, substitutions, omissions, and changes may be made without departing from the spirit thereof. Accordingly, it is intended that the scope of the present invention be limited solely by the scope of the following claims, including equivalents thereof.

1. A method of substantially inhibiting, or reducing mammalian skin irritation, comprising topically applying onto the skin or hair of a candidate subject in need of such treatment, an irritant amount of at least one normally skin-irritating active product and an anti-irritant effective amount of panthenol in admixture with an extract selected from the group consisting of: extract of Centella Asiatica, extract of Calendula, extract of Aesculus Hippocastanum, extract of Ruscus Aculeatus, extract of Glycyrrhiza Glabra and mixtures thereof.

2. The method of substantially inhibiting, or reducing mammalian skin irritation as defined by claim 1, wherein said at least one normally skin-irritating active product is selected from the group consisting of a preservative, surfactant, fragrance, solvent, propellant, biologically active agent and mixtures thereof.

3. The method of substantially inhibiting, or reducing mammalian skin irritation as defined by claim 1, wherein said at least one normally skin-irritating active product is selected from the group consisting of: hair permanent-waving active agent, hair relaxing agent, hair-removing active agent, sunscreen, hydroxy acid, keto acid, retinoid, anthranoid, peroxide, minoxidil, lithium salt, antiproliferating agent, vitamin, hair tint, hair dye, perfuming alcoholic solution, antiperspirant, depigmenting agent, capsaicin, antilouse active agent, ionic detergent agent, nonionic detergent agent, propigmenting agents and mixtures thereof

4. A method of reducing the adverse reaction of skin to chemicals contained in topical cosmetic and pharmaceutical preparations and to contact physical irritants, said method comprising applying to the skin or scalp an anti-irritant effective amount of an anti-irritant composition comprising: panthenol in admixture 0% to 20% by weight of a natural extract selected from the group consisting of: extract of Ruscus Aculeatus, extract of Centella Asiatica, extract of Calendula, extract of Aesculus Hippocastanum, extract of Glycyrrhiza Glabra and mixtures thereof in a carrier suitable for use in contact with the skin, the composition being applied to the skin shortly before, simultaneously with, or shortly after the skin is exposed to a chemical irritant in a cosmetic or pharmaceutical composition or to a contact physical irritant.

5. The method of claim 4 wherein the composition is applied to the skin or scalp simultaneously as the skin is exposed to a chemical irritant or to a contact physical irritant.

6. The method of claim 4 wherein the composition is applied to the skin or scalp simultaneously with a hair permanent-waving active agent.

7. The method of claim 4 wherein the composition is applied to the simultaneously with a hair relaxing active agent.

8. The method of claim 4 wherein the composition is applied to the skin shortly before that skin is waxed for the purpose of removing hair there from.

9. An anti-irritant composition comprising: panthenol in admixture with 0% to 20% by weight an extract selected from the group consisting of: an extract of Ruscus Aculeatus, an extract of Centella Asiatica, an extract of Calendula, an extract of Aesculus Hippocastanum, an extract of Glycyrrhiza Glabra and mixtures thereof, in a carrier suitable for use in contact with the skin.

10. The composition of claim 9 further comprising an ethoxylated sorbitan.

11. The composition of claim 9 further comprising polyvinylpyrrolidone.

12. The composition of claim 9 wherein comprising about 0.5% to 10% by weight of panthenol of the anti-irritant composition.

13. The composition of claim 9 comprising about 0.1 to 20% of at least on extract selected from the group consisting of: the extract of Glycyrrhiza Glabra, the extract of Centella Asiatica, the extract of Calendula, the extract of Aesculus Hippocastanum, the extract of Ruscus Aculeatus and mixtures thereof by weight of the anti-irritant composition.

14. The composition of claim 9 wherein each of: the extract of Centella Asiatica, the extract of Calendula, the extract of Aesculus Hippocastanum, the extract of Glycyrrhiza Glabra and the extract of Ruscus Aculeatus comprises at least about 0.5% by weight of the anti-irritant composition.

15. The composition of claim 9 admixed with a cosmetic or pharmaceutical composition containing a chemical irritant.

16. The composition of claim 9 admixed with a hair relaxer composition.

17. The composition of claim 9 admixed with a hair permanent or waving composition.
